(19) United States
(12) Patent Application Publication
(10) Pub. No.: US 2008/0108954 A1
(43) Pub. Date: May 8, 2008

(54) Flow Controllers

(76) Inventors: Jean-Marie Mathias, Lillois (BE); Richard L. West, Lake Villa, IL (US); Georges Rondeau, Brafee (BE); Joseph H. Bowman, Lake Villa, IL (US); Benjamin S. Grant, Ingleside, IL (US); Daniel F. Bischof, Bull Valley, IL (US)

Correspondence Address: BAXTER HEALTHCARE CORPORATION, ONE BAXTER PARKWAY, DF2-2E, DEERFIELD, IL, 60015, US

(21) Appl. No.: 11/555,797

(22) Filed: Nov. 2, 2006

(51) Int. Cl. A61M 39/22

(52) U.S. Cl. 604/248; 604/246; 604/500

(57) ABSTRACT

Flow controllers are provided for interconnecting a fluid source to two separate collection zones. The flow controllers include a body having a fluid inlet communicating with the source and two fluid outlets, each communicating with one of the collection zones. An actuator member received within a cavity of the body includes first and second flow paths. In a first position, the first flow path is aligned with the fluid inlet of the body to allow fluid communication with one of the fluid outlets and the associated collection zone. The actuator member is non-rotationally moved to a second position within the cavity to align the second flow path with the fluid inlet of the body, thereby halting flow through the first flow path and allowing fluid communication with the other collection zone. An optional safety feature prevents movement of the actuator member from the second position to the first position.

### BACKGROUND

1. Field of the Disclosure

This disclosure generally relates to apparatus for controlling fluid, such as in (but not limited to) the collection of blood from a donor, in particular blood collected in at least two separate containers. More particularly, the disclosure relates to valves suitable for switching blood flow between first and second blood collection containers. Even more particularly, this disclosure relates to directing initial blood flow from a donor to a first container and irreversibly diverting the blood flow to a second container.

2. Description of Related Art

A disposable plastic container and tubing set or fluid circuit is typically used for collecting blood from a donor. The disposable blood collection set includes a venipuncture needle for insertion into the arm of the donor. The needle is attached to one end of a flexible plastic tube which provides a flow path for the blood. The flow path communicates with one or more plastic bags or containers for collecting the withdrawn blood.

The blood collection set may also include a sampling sub-unit The sampling sub-unit allows for collection of a sample of blood, which sample can be used for testing of the blood. Preferably, the sample is obtained prior to the “main” collection of blood. Collecting the sample prior to the main collection reduces the risk that bacteria residing on the donor's skin where the needle is inserted (i.e., in particular, the small section of detached skin commonly referred to as the “skin plug”) will enter the collection container and contaminate the blood collected for transfusion. Thus, it is preferred that the blood sample, which may include the skin plug, be diverted from the main collection container.

Examples of blood collection sets with such a “pre-donation” sampling sub-unit are described in U.S. Pat. Nos. 6,387,086 and 6,520,948 and in U.S. Patent Application Publication Nos. 2005/0215975 and 2005/0148993, all of which are hereby incorporated herein by reference. The collection sets described therein are generally illustrated in FIG. 1 at 10 and include a needle (not illustrated) and a length of tubing 12, defining a flow path, one end of which communicates with the needle and the other end of which communicates with the inlet port 14 of a Y-junction 16. The tubing set also includes two additional lines 18 and 20 which are branched from the outlet ports 22 and 24 of the Y-junction 16, respectively. The first branched line 18 is attached to a sample pouch 26 for collecting a smaller volume of blood from which samples may be obtained. Typically, approximately 50 ml of blood is a sufficient amount to provide an adequate sample size and to clear the skin plug from the tubing set. The second branched line 20 is attached to a main collection container 28 that is typically adapted to collect a larger quantity of blood than the sample pouch 26 after the initial sample has been taken.

The blood collection set 10 of FIG. 1 also includes flow control clamps 30, 32 for controlling the flow of biological fluid (e.g., blood) through the set. The three ports of the Y-junction 16 are always open, so the tubing associated with each must include separate means for regulating flow therethrough. Flow control clamps commonly used are the Roberts-type clamps, which are well known in the art. Clamps of this type are generally described in U.S. Pat. Nos. 3,942,228; 6,089,527; and 6,113,062, all of which are hereby incorporated herein by reference. The clamp described in U.S. Patent Application Publication No. 2005/0215975 may instead be used in operations where it is desirable to irreversibly close flow through a length of tubing.

The clamps 30, 32 are typically placed on the tubing line 12 leading to the Y-junction 16 and on the tubing line 18 leading to the sample pouch 26, respectively. A clamp may also be placed on the tubing line 20 leading to the main collection container 28, but flow through that tubing line 20 is frequently regulated by a breakaway cannula 34, as illustrated in FIG. 1. By selectively opening and closing the different flow paths (by depressing or releasing the clamps), the technician can control the flow of blood from the donor, diverting the blood to the desired output zone.

In a typical application, the clamp 30 on the initial length of tubing 12 is closed and venipuncture is performed on the donor. Thereafter, the clamps 30 and 32 are opened to allow a small amount of blood to be collected in the sample pouch 26 for later analysis and to clear the skin plug. When the desired amount of blood has been collected in the sample pouch 26, the clamp 32 between the Y-junction 16 and the sample pouch 26 is closed and the breakaway cannula 34 is broken to allow blood flow to the main collection container 28. Flow to the sample pouch 26 should be permanently closed, in order to prevent the skin plug from migrating into the main collection container 28 and to prevent anticoagulant from migrating to the sample pouch 26 from the main collection container 28.

Clearly, the above-described process involves several steps and the manipulation of a number of different components. Accordingly, there have been attempts to provide flow controllers that simplify the blood sample collection process, while avoiding contamination by a skin plug. For example, U.S. Pat. No. 6,626,884 to Dillon et al., which is hereby incorporated herein by reference, describes a number of devices and methods for pre-donation blood sample collection. The described devices include at least four positions: (1) a sampling position for collecting a sample and clearing the skin plug, (2) a collecting position for collecting a larger amount of blood in one or more collection bags, (3) an intermediate closed position between the first two positions for preventing both sampling and collection, and (4) a final closed position beyond the collecting position for finally closing flow through the device. One possible drawback of such devices is that a minimum amount of skill and training may be required for a user to recognize the various positions and properly manipulate the device. Furthermore, if the device is maintained in the intermediate closed position for an extended period of time, then blood in the inlet line may begin to coagulate before being transferred to the collection bags, leading to a number of known problems.

U.S. Pat. No. 6,692,479 to Kraus et al., which is hereby incorporated herein by reference, discloses another example of a flow controller useful in the collection of pre-donation blood samples. The flow controller described therein includes inlet and outlet flow members, wherein one of said members is arranged for rotation about an axis to align an inlet port with a selected outlet port. While the controller reduces the number of operator steps required (as compared to systems that utilize clamps and frangible devices), it likely requires two-handed operation by the operator and some skill and training to properly manipulate the device.

Therefore, there is still a need for improved flow controllers that reduce the components of known blood collection sets and reduce the number of steps that the operator is required to remember and perform, thereby simplifying the process of collecting separate amounts of blood.

### SUMMARY

There are several aspects of the present invention which are embodied in the devices, systems and methods described and claimed below.

Accordingly, in one aspect, a flow controller is provided with a body defining a cavity. The body includes a fluid inlet, a first fluid outlet, and a second fluid outlet. An actuator member is at least partially received within the cavity and defines a first flow channel and a second flow channel. The actuator member is adapted for at least substantially non-rotational movement from a first position to a second position within the cavity. In the first position, the first flow channel allows for fluid communication between the fluid inlet and the first fluid outlet. In the second position, the second flow channel allows for fluid communication between the fluid inlet and the second fluid outlet. The actuator member is prevented from moving from the second position to the first position.

In another aspect, a flow controller is provided with a body defining a cavity. The body includes a fluid inlet, a first fluid outlet, and a second fluid outlet. A generally cup-shaped insert is received within the cavity and has an inlet hole aligned with the fluid inlet, a first outlet hole aligned with the first fluid outlet, and a second outlet hole aligned with the second fluid outlet. An actuator member is at least partially received within the insert for movement from a first position to a second position within the insert. In the first position, the actuator member allows for fluid communication between the fluid inlet and the first fluid outlet. In the second position, the actuator member allows for fluid communication between the fluid inlet and the second fluid outlet.

In accordance with yet another aspect, a fluid processing set is provided with first and second collection containers and a flow controller. The flow controller has a body defining a cavity. The body includes a fluid inlet, a first fluid outlet communicating with the first collection container, and a second fluid outlet communicating with the second collection container. An actuator member is at least partially received within the cavity and defines a first flow channel and a second flow channel. The actuator member is adapted for at least substantially non-rotational movement from a first position to a second position within the cavity. In the first position, the first flow channel allows for fluid communication between the fluid inlet and the first fluid outlet. In the second position, the second flow channel allows for fluid communication between the fluid inlet and the second fluid outlet. The actuator member is prevented from moving from the second position to the first position.

In another aspect, a method of collecting at least two quantities of a biological fluid from a biological fluid source involves providing a first collection container, a second collection container, a flow controller body, and an actuator member. The flow controller body has a fluid inlet, a first fluid outlet communicating with the first collection container, and a second fluid outlet communicating with the second collection container. The actuator member defines a first flow channel and a second channel separate from the first channel, and is movably received by the body. Fluid flow is introduced to the fluid inlet of the flow controller body with the actuator member in a first position within the flow controller body, thereby directing the flow through the first flow channel and the first fluid outlet to the first collection container. Thereafter, the actuator member is moved from the first position to a second position within the flow controller body without substantial rotational movement, thereby directing the blood flow through the second flow channel and the second fluid outlet to the second collection container. The actuator member is prevented from moving to the first position from the second position.

Flow controllers and methods generally described herein are particularly well-suited for use in connection with a blood sample collection set to isolate an initial quantity of blood from the main collection quantity. However, flow controllers and methods according to the present invention are not limited to use with specific fluids or collection processes and may be applied to virtually any flow system requiring switching, preferably irreversibly, between at least two output zones.

### BRIEF DESCRIPTION OF THE DRAWINGS

FIG. 1 is a schematic view of a known blood collection set;

FIG. 2 is a schematic view of a blood collection set incorporating a flow controller according to an aspect of the present invention;

FIG. 3 is a front perspective view of a flow controller suitable for use in the blood collection set of FIG. 2, in a first position;

FIG. 4 is a front perspective view of the flow controller of FIG. 3, in a second position;

FIG. 5 is a front perspective view of a body of the flow controller of FIG. 3;

FIG. 6A is a front perspective view of an actuator member of the flow controller of FIG. 3;

FIG. 6B is a rear perspective view of the actuator member of FIG. 6A;

FIG. 7 is a front perspective cross-sectional view of the flow controller of FIG. 3, taken through the line 7-7 of FIG. 3;

FIG. 8 is a front perspective cross-sectional view of the flow controller of FIG. 4, taken through the line 8-8 of FIG. 4;

FIG. 9 is a front perspective exploded view of a flow controller incorporating an insert between the body and the actuator member;

FIG. 10 is a front perspective exploded view of another embodiment of a flow controller incorporating an insert between the body and the actuator member;

FIG. 11 is a front perspective exploded view of yet another embodiment of a flow controller incorporating an insert between the body and the actuator member;

FIG. 12 is a front perspective view of the body of the flow controller of FIG. 11;

FIG. 13 is a rear perspective view of the actuator member of the flow controller of FIG. 11;

FIG. 14A is a front perspective assembled view of the flow controller of FIG. 11, in a first position;

FIG. 14B is a cross-sectional view of the flow controller of FIG. 14A, taken through the line 14C-14C of FIG. 14A;

FIG. 14C is another cross-sectional view of the flow controller of FIG, 14A, taken through the line 14C-14C of FIG. 14A;

FIG. 15A is a front perspective assembled view of the flow controller of FIG. 11, in a second position;

FIG. 15B is a cross-sectional view of the flow controller of FIG. 15A, taken through the line 15C-15C of FIG. 15A;

FIG. 15C is another cross-sectional view of the flow controller of FIG. 15A, taken through the line 15C-15C of FIG. 15A;

FIG. 16 is a front perspective view of an alternative actuator member suitable for use with flow controllers according to the present invention;

FIG. 17 is a front perspective view of an alternative insert suitable for use with flow controllers according to the present invention;

FIG. 18 is a front elevational view of the actuator member of FIG. 16 received in the insert of FIG. 17, in a first position;

FIG. 19 is a front perspective exploded view of another embodiment of a flow controller according to an aspect of the present invention;

FIG. 20 is a front perspective exploded view of an alternative actuator member suitable for use with the flow controller of FIG. 19;

FIG. 21 is a front perspective assembled view of the actuator member of FIG. 20;

FIG. 22 is a front elevational view of the actuator member of FIG. 21 received in an insert, in a first position;

FIG. 23 is a front elevational view of the actuator member of FIG. 21 received in an insert, in a second position;

FIG. 24 is an exploded view of a flow controller according to another embodiment of the present invention;

FIG. 25 is a cross-sectional view of the flow controller of FIG. 24, in a first position;

FIG. 26 is a cross-sectional view of the flow controller of FIG. 24, in a second position; and

FIG. 27 is a front perspective view of a substantially non-cylindrical actuator member.

### DESCRIPTION OF THE PREFERRED EMBODIMENTS

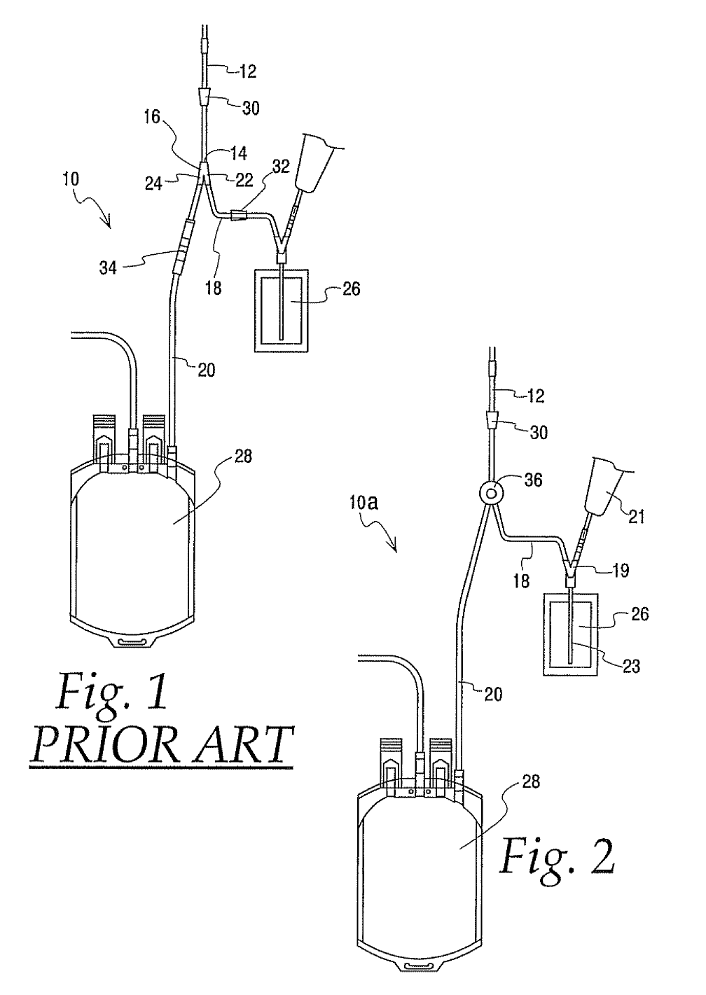

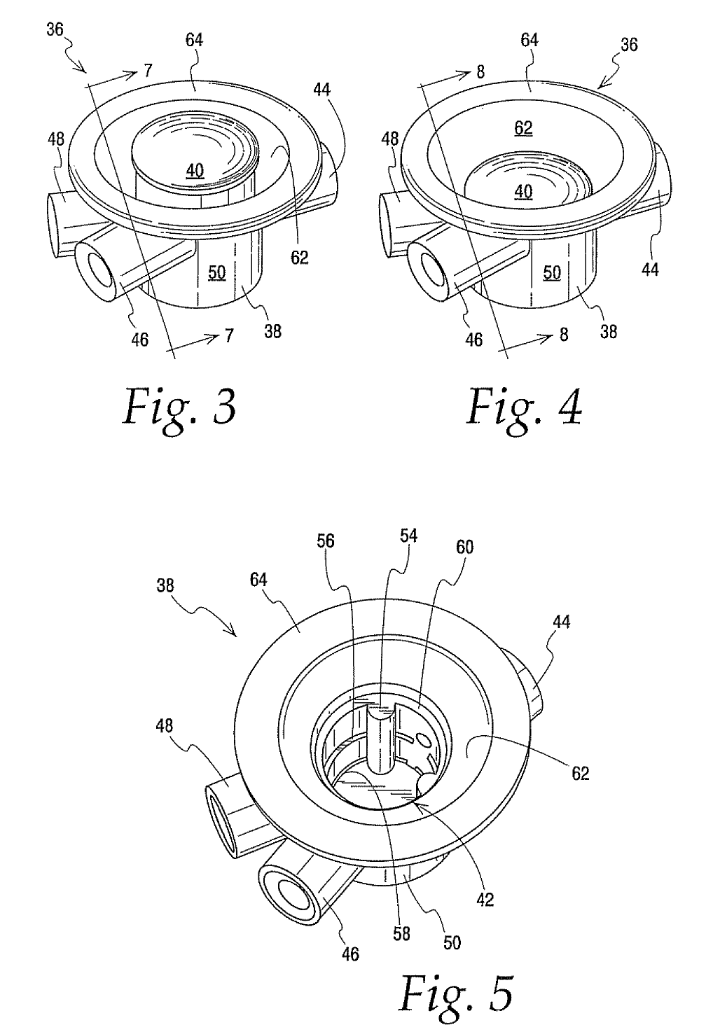

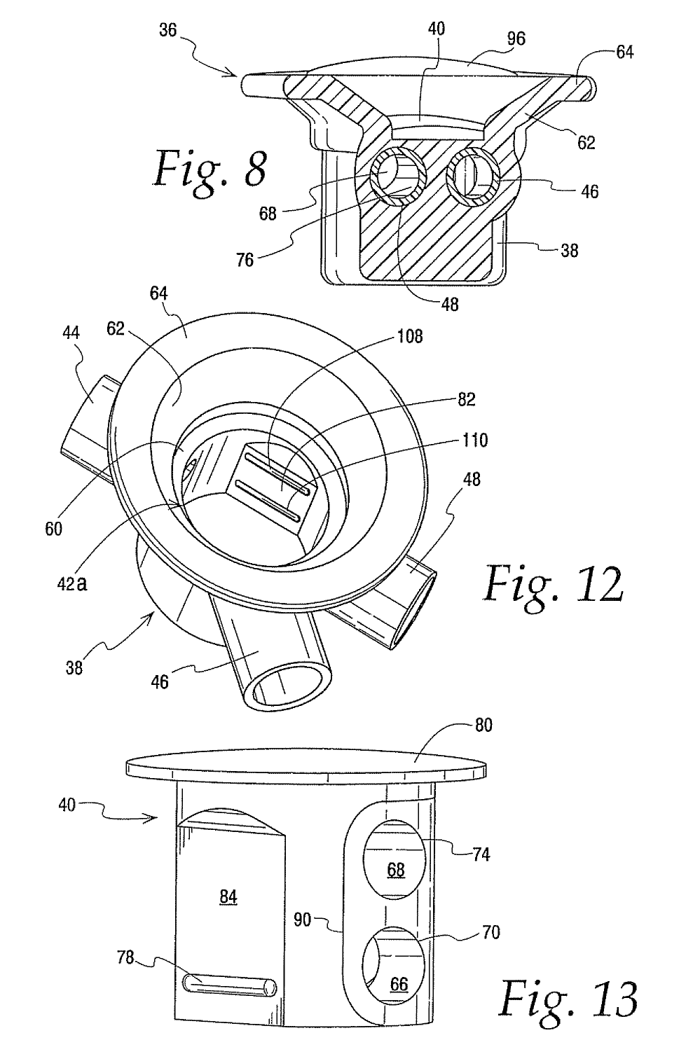

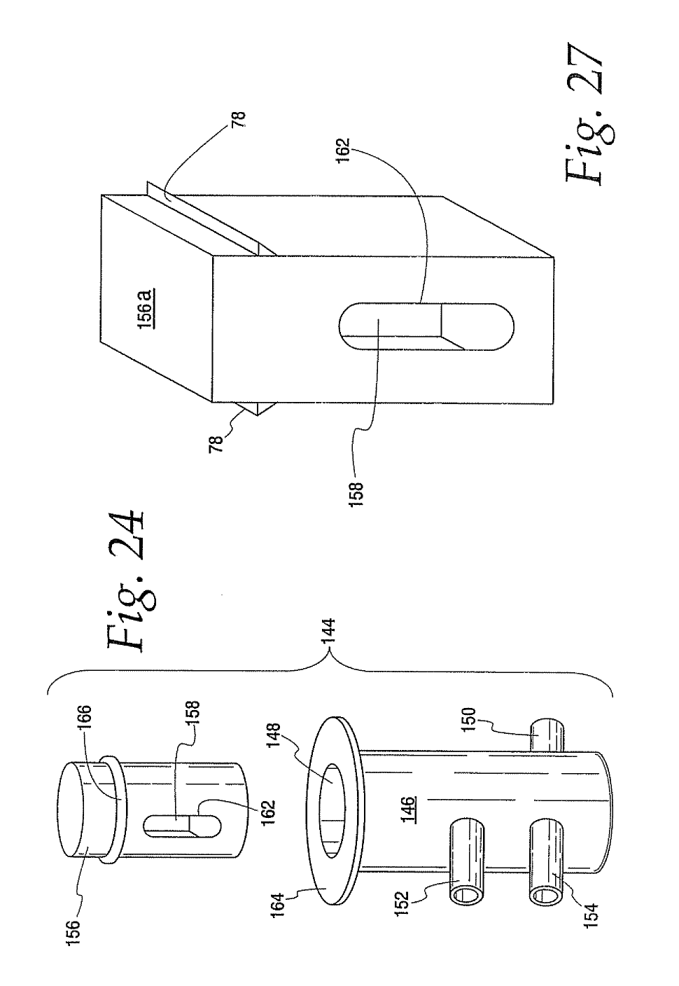

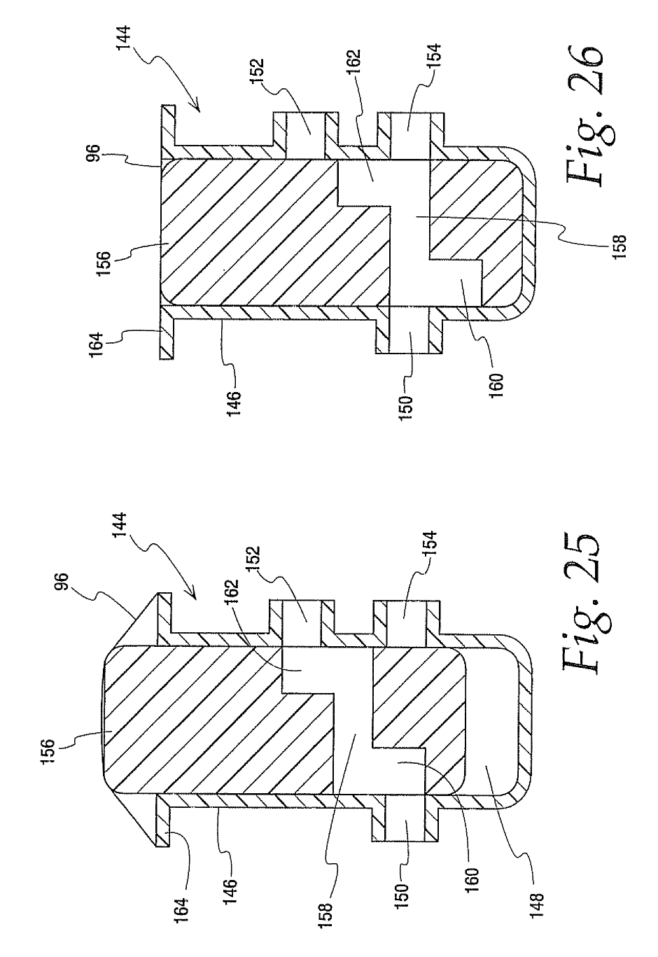

It will be seen from the following description that there are several possible variations and embodiments of flow controllers according to the present invention, including the flow controllers generally shown in FIGS. 1-23 and the flow controllers shown in FIGS. 24-27. Common to all of the embodiments described and shown below is a flow controller having a body (e.g., element 38 of FIG. 3 and element 146 of FIG. 24) with a fluid inlet (e.g., element 44 of FIG. 3 and element 150 of FIG. 24) and first and second fluid outlets (e.g., elements 46 and 48, respectively, of FIG. 3 and elements 152 and 154, respectively, of FIG. 24). The body is adapted to receive an actuator member (e.g., element 40 of FIG. 3 and element 156 of FIG. 24). As will be described in further detail below, the actuator member is further adapted for movement within the body to selectively bring the fluid inlet into communication with the first fluid inlet or second fluid outlet. The actuator is adapted for at least substantially non-rotational movement and more preferably no rotational movement between first and second positions, as generally shown in FIGS. 25-26 (and FIGS. 7-8) within the body. As used herein “substantially non-rotational” means no more than de-minimis movement of the actuator about a central axis. “Substantially non-rotational” movement falls short of a rotational movement that would allow an inlet to be in flow communication with an outlet.

In the first position, the actuator provides for fluid communication between the fluid inlet and the first fluid outlet, but not the second fluid outlet. In the second position, the actuator provides for fluid communication between the fluid inlet and the second fluid outlet. Once in the second position, a common feature of all of the embodiments disclosed herein is that the actuator member is prevented from moving from the second position to the first position.

Flow controllers embodying the principles described herein are simple to operate, as they may be actuated with one hand and involve only a button press. Simplifying the process also makes it more reliable, because the user cannot inadvertently misalign or otherwise obstruct flow through the system. To further enhance safety when using the described flow controllers in a blood sample collection kit or the like, they may be adapted for one-time, one-way operation, which prevents return movement from a final position to an initial backflow, thereby eliminating the risk of upstream or downstream contamination. Flow controllers described herein also maintain sterility of the system by providing a sanitary seal (referenced by numeral 96 in the figures) over the actuator. Further details and preferred embodiments of the above-described flow controller are set forth below.

It will be understood that the disclosed embodiments generally described below and illustrated in the attached drawings are merely exemplary of the present invention, which may be embodied in various forms. Therefore, specific details disclosed herein are not to be interpreted as limiting, but rather as representative and provide a basis for variously employing the present invention in any appropriate manner understood by one of ordinary skill in the art.

All aspects of the flow controllers described herein and, in particular, the illustrated embodiments which follow may be adapted to cooperate with conventional tubing and blood collection sets.

FIG. 2 shows a blood collection set 10a incorporating a flow controller or valve 36 according to an aspect of the present invention The components of the blood collection set 10a that are common to the blood collection set 10 of FIG. 1 are identified with the same reference numerals. Thus, collection set 10a includes a venipuncture needle (not shown) and a tube 12 defining a flow path, one end of which communicates with the needle. The other end of tube or line 12 is attached to an inlet of flow controller 36 which will be described in greater detail below. One end of line or tube 18 is attached to an outlet of flow controller 36. The other end of tube 18 is joined to an access site 19. As shown in FIG. 2, access site 19 may typically be a Y-type access site, with an end of tube 18 communicating with one leg or portion of access site 19. The other leg or adjacent portion of the Y-type access site may be adapted for receiving a tube holder 21 for receiving vacuum sealed sample tubes. The tube holder 21 may be preattached to access site 19 or may be separately provided, as shown and described in U.S. Patent Application Publication No. 2005/0148993, previously incorporated by reference.

Sample pouch 26 may also include an internal flow path 23 that extends substantially into pouch 26 and one end of which also communicates with access site 19. Preferably, as described in U.S. Patent Application Publication No. 2005/0148993, and also shown in U.S. Pat. Nos. 6,387,086 and 6,520,948 (see FIG. 2D), flow path 23 is the only flow path whereby blood for sampling enters and exits the internal chamber of pouch 26.

It will be seen that the blood collection set 10a is simplified with respect to the blood collection set 10 of FIG. 1, because there is no need for clamps and/or breakaway cannulas on the tubing 18, 20 leading to the main collection container 28 and the sample pouch 26. This reduction in parts decreases the cost and complexity of assembling the blood collection set 10a and, as described in greater detail herein, simplifies the blood collection process. However, while the flow controllers according to the present invention are suitable for use with blood collection sets according to the above description, they are generally applicable to any fluid transfer system requiring the non-simultaneous transfer of a fluid from a single source to at least two output locations.

Turning now more particularly to the flow controller 36, FIGS. 3-8 illustrate a first embodiment. The flow controller 36 includes a body 38 and an actuator member 40 movably received by a cavity 42 of the body 38. The illustrated body 36 includes a fluid inlet 44, a first fluid outlet 46, and a second fluid outlet 48. The fluid inlet 44 and the fluid outlets 46 and 48 may, as shown in FIGS. 3 and 4, have the same vertical elevation, effectively defining a “flow plane” through the flow controller 36. The fluid inlet 44 and the fluid outlets 46 and 48 are preferably adapted for connection with flexible tubing according to known construction. The fluid inlet 44 is communicable with a fluid source, typically a phlebotomy needle, while the fluid outlets 46 and 48 are communicable with separate collection zones, preferably a sample pouch and a main collection container, respectively.

The body 38 is illustrated with two fluid outlets 46 and 48 separated by an angle generally bisected by an axis of the fluid inlet 44, but a number of other orientations are possible, two of which are shown in FIGS. 10 and 11. The embodiment of FIG. 10 has three substantially parallel, non-coaxial coaxial ports (the fluid inlet is not visible, but defines an axis parallel to and midway between the fluid outlets 46 and 48) and the embodiment of FIG. 11 has a straight flow path defined by the inlet port 44 and the second outlet port 48, and a branch or leg defined by the first outlet port 46. The orientation of FIG. 11 may be preferred because it includes a second fluid outlet 48 coaxial with the fluid inlet 44, which simplifies manufacture of the body 38 and minimizes the risk of flow stagnation through the second fluid outlet 48, as will be described herein. Furthermore, although the illustrated fluid inlets and outlets define a “flow plane” extending through a sidewall 50 of the body 38, it will be appreciated from the following description that the present invention may be practiced with a flow controller having any one of the fluid inlet and the fluid outlets positioned at a bottom surface of the body or at a different vertical elevation (not illustrated). Additionally, the body may be provided with more than two fluid outlets without departing from the scope of the present invention.

As best illustrated in FIG. 5, the body 38 defines an open-top cavity 42 in communication with the fluid inlet 44 and fluid outlets 46 and 48 through the sidewall 50. The cavity 42 of FIG. 5 includes at least one vertical post 54 and at least two horizontal arcuate grooves 56 and 58. Preferably, the top of the cavity is bounded by an annular seat 60 with a funnel-shaped upper wall 62 that terminates at an annular sealing surface 64. The function of the vertical post 54, the horizontal grooves 56 and 58, the seat 60, and the sealing surface 64 will be explained in greater detail herein.

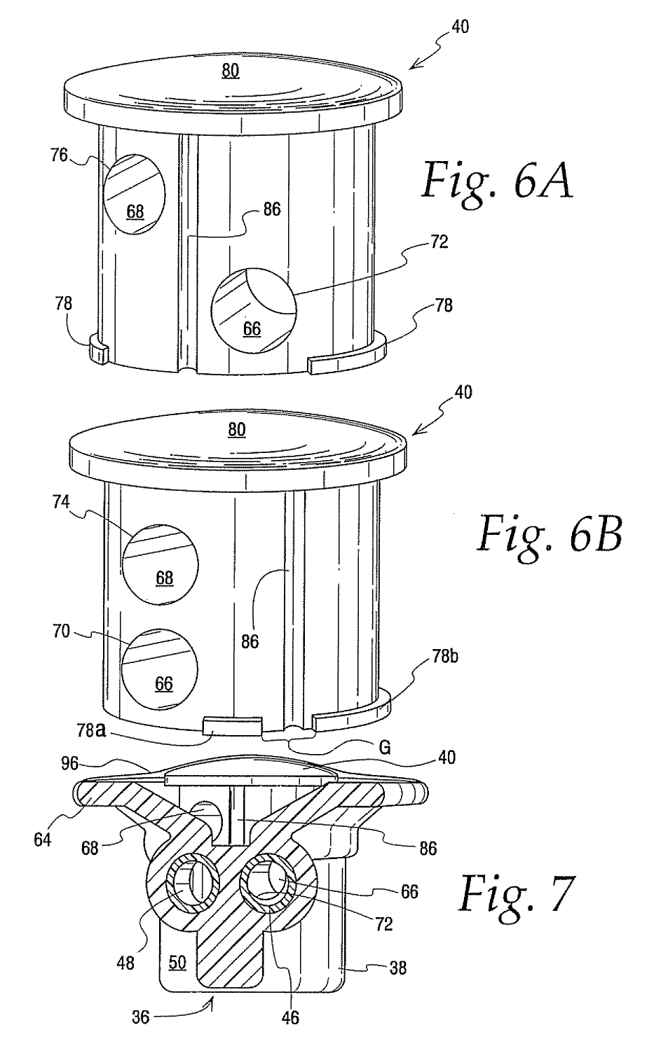

The cavity 42 is adapted to receive an actuator member or button 40, illustrated in detail in FIGS. 6A and 6B. The actuator member 40 includes a plurality of flow paths or channels, which are not in fluid communication with each other. Preferably, the actuator member 40 is provided with a separate flow path corresponding to each fluid outlet 46, 48 of the body 38. Hence, the illustrated actuator member 40 includes a first or lower flow path 66 and a second or upper flow path 68 extending therethrough. The lower flow path 66 extends from a lower fluid entrance 70, shown in FIG. 6B, to a lower fluid exit 72, shown in FIG. 6A. Similarly, the upper flow path 68 extends from an upper fluid entrance 74 (FIG. 6B) to an upper fluid exit 76 (FIG. 6A). The actuator member 40 may be comprised of a rigid, non-compressible material to eliminate any risk of it deforming and thereby restricting flow through the flow paths 66 and 68,

The actuator member 40 is preferably initially provided in a first position, illustrated in FIGS. 3 and 7, wherein the lower fluid entrance 70 is aligned with the fluid inlet 44 (not visible in FIG. 7) of the body 38 and the lower fluid exit 72 is aligned with the first fluid outlet 46 of the body 38, thus allowing fluid communication between the fluid inlet 44 and the first fluid outlet 46 through the lower flow path 66. As illustrated in FIG. 7, fluid flow through the second fluid outlet 48 of the body 38 is closed in the first position, because the upper flow path 68 is not aligned with the fluid inlet 44.

To maintain the actuator member 40 in the first position, it is preferably provided with one or more radially projecting ribs or latches 78 (FIGS. 6A and 6B) adapted to seat within the upper groove 56 of the body cavity 42 (FIG. 5). If the latches 78 are spaced about the lower perimeter of the actuator member 40, as shown in FIG. 6A, then the actuator member 40 will sit level in the upper groove 56 and resist “rocking” when moved to a second position, as will be described in greater detail herein. Of course, the placement of the latches 78 and grooves 56, 58 may be reversed, with a groove on the actuator member and inwardly projecting latches on the body cavity (not illustrated). However, such an embodiment may not be preferred because it may be more difficult to form such structures during manufacture.

To institute fluid flow between the fluid inlet 44 and the second fluid outlet 48 of the body 38, the actuator member 40 is advanced further into the body cavity 42, or downwardly in terms of the orientation of FIGS. 3 and 4, to a second position shown in FIGS. 4 and 8 In the second position, the upper fluid entrance 74 is aligned with the fluid inlet 44 of the body 38 and the upper fluid exit 76 is aligned with the second fluid outlet 48 of the body 38, thus allowing fluid communication between the fluid inlet 44 and the second fluid outlet 48 through the upper flow path 68. As illustrated in FIG. 8, fluid flow through the first fluid outlet 46 is closed in the second position. Thus, it will be seen from the preceding description that the actuator member 40 is adapted to move through a linear path at an angle to the “flow plane,” preferably perpendicularly thereto

To maintain the actuator member 40 in the second position, the latches 78 move from the upper groove 56 of the body cavity 42 and into the lower groove 58. The latches 78 may be provided with a flat, outwardly extending top surface that interacts with the lower groove 58 like a ratchet pawl to prevent movement from the second position to the first position. To prevent the actuator member 40 from moving past or overshooting the second position, it may be provided with an oversized endcap 80 that contacts and interferes with the seat 60 of the body cavity 42 to prevent further advancement of the actuator member 40 into the cavity 42. Alternatively or additionally, the bottom surface of the actuator member may be adapted to contact the bottom surface of the cavity in the second position to prevent further advancement of the actuator member into the cavity.

The actuator member 40 is linearly advanced from the first position to the second position within the cavity 42. If the actuator member 40 is allowed to rotate with respect to the cavity 42, then the flow paths 66 and 68 will become misaligned and performance may degrade. According to one manner of preventing rotation, the latch 78 may be provided with a break or gap “G” (FIG. 6B) adapted to receive the vertical post 54 of the cavity 42. The break “G” has substantially the same width as the vertical post 54, such that first and second portions 78a and 78b of the latch 78 act as lateral barriers that bear against the vertical post 54 when a user attempts to rotate the actuator member 40.

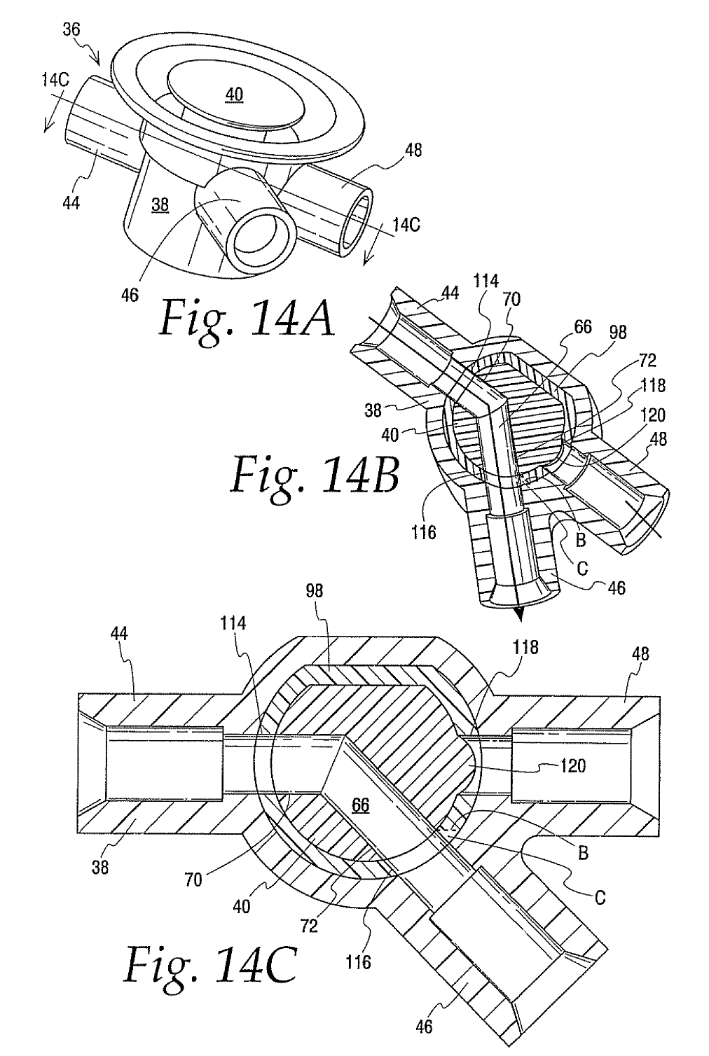

According to another manner of preventing rotation, the body 38 and actuator member 40 may each be provided with flat walls 82 and 84, respectively, as shown in FIGS. 12 and 13. The non-cylindrical cavity 42a resulting from the flat wall 82 will only receive the actuator member 40 in one orientation, i.e., one in which the flat walls 82 and 84 are aligned. Such a keying relationship prevents rotation of the actuator member 40, thereby ensuring that the proper alignment is maintained between the various components of the actuator member 40 and the various components of the body 38.

The actuator member 40 may provide a relatively tight fit with the cavity 42, 42a in order to prevent leakage at the actuator member-body interfaces, for example leakage from the first fluid outlet 46 when the actuator member 40 is in the second position (FIG. 8). In such an embodiment, the actuator member 40 may be provided with a vent channel 86 (FIGS. 6A-7) to vent any air trapped between the actuator member 40 and the cavity 42, 42a during movement to the second position.

According to another embodiment, a plateau 88 extending slightly radially beyond the curved wall of the actuator member 40 may be provided about the fluid exits 72 and 76 (FIG. 11) to more closely conform to the region of the cavity 42, 42a adjacent to the fluid outlets 46 and 48. A separate plateau 90 may be provided about the fluid entrances 70 and 74 (FIG. 13) to create a tighter fit with the region of the cavity 42, 42a adjacent to the fluid inlet 44. An additional benefit of the plateau 88, 90 is that the remainder of the curved wall of the actuator member 40 is slightly offset from the cavity 42, 42a, thereby providing ventilation of any air trapped between the actuator member 40 and the cavity 42, 42a during movement to the second position, thereby eliminating the need for a separate vent channel 86.

The actuator member 40 may be comprised of any of a number of materials. For example, in one embodiment, the actuator member is relatively rigid or non-compressible, and comprised of a material such as polypropylene. It may be preferred to use a rigid actuator member, because such an embodiment provides a more secure fit with the cavity grooves and an improved tactile and/or audible indication when moved to the second position. In particular, the latch may make a “clicking” noise when it snaps into place in the groove of the body. This is merely one possible indicating means and those of ordinary skill in the art will recognize that others are available and may be practiced with this aspect of the present invention.

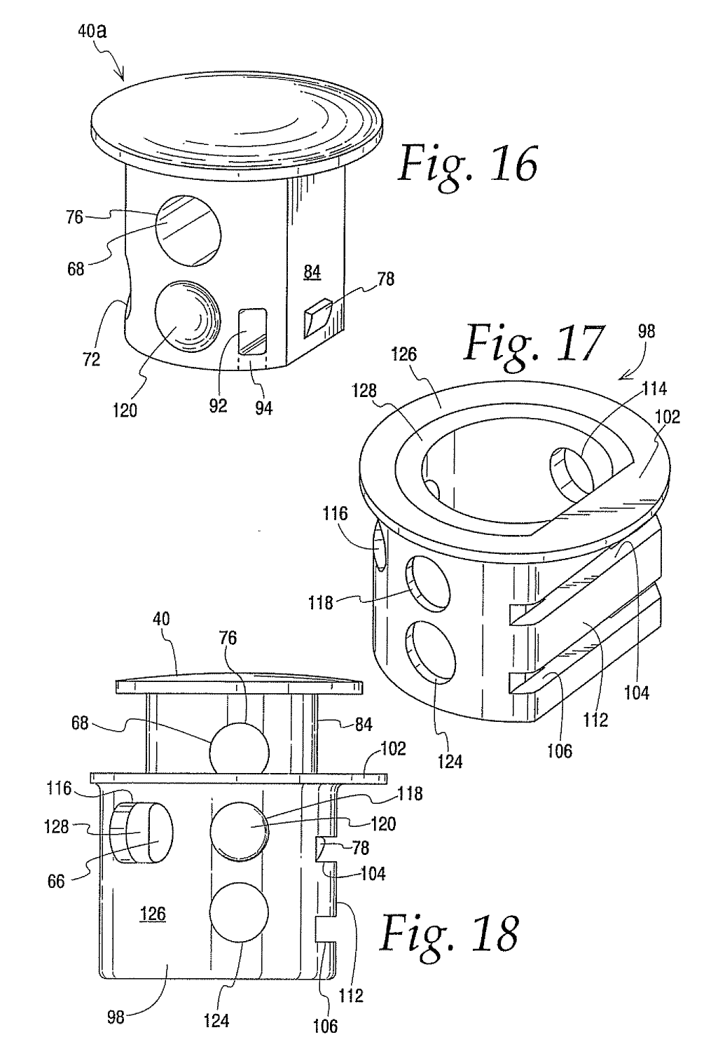

FIG. 16 illustrates an actuator member design suitable for use with a rigid material. In contrast to the actuator member 40 of FIGS. 6A and 6B, the actuator member 40a includes a through hole 92 generally adjacent to the latch 78. The through hole 92 weakens the surrounding area and allows the latch 78 to be deformed slightly inwardly when the actuator member 40a is moved from the first position to the second position. When the latch 78 moves into the vicinity of the lower groove 58 (FIG. 5), it resiliently returns to the undeformed orientation to lock in place. Alternatively, if additional deformation is required of the latch 78, then the area beneath the through hole 92 (illustrated in broken lines at 94 in FIG. 16) may be removed to make the latch 78 even more pliable.

Alternatively, the actuator member 40 may be comprised of a less rigid, more deformable material. A more deformable actuator member is less dependent on precise manufacturing tolerances than a more rigid one, and may be better suited to providing a leak-resistant fit against the body cavity 42, 42a. On the other hand, the actuator member should not be overly deformable, otherwise it will deform when pressed, instead of moving to the second position. Further, a latch 78 made of an overly deformable material may be insufficient to lock into a groove 58 to prevent movement from the second position to the first position. It has been found that an actuator member having a Shore hardness rating of approximately 80 will function properly, without suffering from any of the above drawbacks. In particular, suitable materials include Cawiton SEBS, manufactured by Wittenburg B.V. of Hoevelaken, Netherlands, and Santoprene® thermoplastic elastomer, manufactured by Advanced Elastomer Systems, LP of Akron, Ohio. These materials are especially suitable for use with a relatively rigid body formed of polycarbonate, because they will not become bonded thereto if the flow controller is subjected to a steam sterilization process at approximately 240° F.

When practiced with a blood sample collection set 10a according to FIG. 2, the first fluid outlet 46 may communicate with the sample pouch 26 and the second fluid outlet 48 may communicate with the main collection container 28. As illustrated, the flow controller 36 allows for the elimination of the clamp 32 on the sample pouch tubing line 18 and the cannula 34 on the collection line 20 (FIG. 1). As a result, the blood sample collection set 10a is less expensive to manufacture and simpler to operate.

Contamination of the fluid, especially if the fluid is blood, should be prevented, so the body 38 may be provided with a sanitary seal or membrane 96 bonded to the annular sealing surface 64 that covers the cavity 42 and encloses the actuator member 40 (FIGS. 7 and 8). The membrane 96 is not illustrated in certain other embodiments for purposes of clarity, but it should be understood that any flow controller according to the present invention may be provided with a sealing membrane to prevent contamination during use. Preferably, the membrane 96 is sufficiently deformable to flex and allow the actuator member 40 to be moved from the first position to the second position. Polyvinyl chloride (PVC) is a suitable material for the membrane 96 and may be RF heat-sealed to the body 38, but other materials may be used without departing from the scope of the present invention.

Another concern is preventing stagnation of the fluid as it passes through the flow paths 66 and 68 of the actuator member 40, 40a. If blood is allowed to stand, then it may coagulate, leading to a number of well-known sample collection problems. If the actuator member 40, 40a may be moved to an intermediate position, between the first and second positions, then the blood in the first flow path 66 can become trapped therein, risking coagulation. In order to avoid this risk, the first and second positions may be relatively close together, with a total button stroke in the range of approximately 0.15 inch and approximately 0.16 inch. Such a button stroke makes it difficult for a user to inadvertently establish an intermediate position between the intended first and second positions. Additionally, the actuator member 40, 40a and body 38 may be adapted such that there is no closed intermediate position, but instead an intermediate position allowing for some nominal cross-talk between the fluid outlets 46 and 48 instead.

It has also been found that requiring blood to change directions, i.e. move through a non-linear flow path, risks stagnation and coagulation. Accordingly, a body having a coaxial fluid outlet 48 according to FIG. 11 may be preferred, with the first fluid outlet 46 being associated with a sample pouch 26 (FIG. 2) and angled with respect to the fluid inlet 44, and the second fluid outlet 48 being associated with a main collection container 28 (FIG. 2) and coaxial with the fluid inlet 44. As described herein, only a small amount of blood is sent to the sample pouch 26, whereas a greater amount of blood is sent to the main collection container 28. Accordingly, the body 40 of FIG, 11 minimizes the risk of coagulation by associating the angled fluid outlet 46 with the sample pouch 26 and the coaxial fluid outlet 48 with the main collection container 28.

To further promote a sanitary collection environment, the flow controller itself may be sterilized prior to use. Preferably, the body and actuator member are irradiated and steam sterilized during manufacture to ensure that the flow controller and associated tubing and containers are sterile. One possible problem with steam sterilization, which may be carried out at approximately 240° F., is that the heat may tend to cause the body to deform, thereby degrading performance. For example, in one embodiment, the body is formed of PVC, which is useful for bonding to PVC tubing and a PVC sealing membrane, but can shrink and deform during steam sterilization. While it is within the scope of the present invention to use a more rigid material, such as polycarbonate or stainless steel, doing so may lead to other problems, such as increased complexity of properly sealing tubing to the fluid inlet and outlets, and the risk of the body inadvertently becoming bonded to other components, such as the sample pouch or main collection container, during manufacturing and/or packaging.

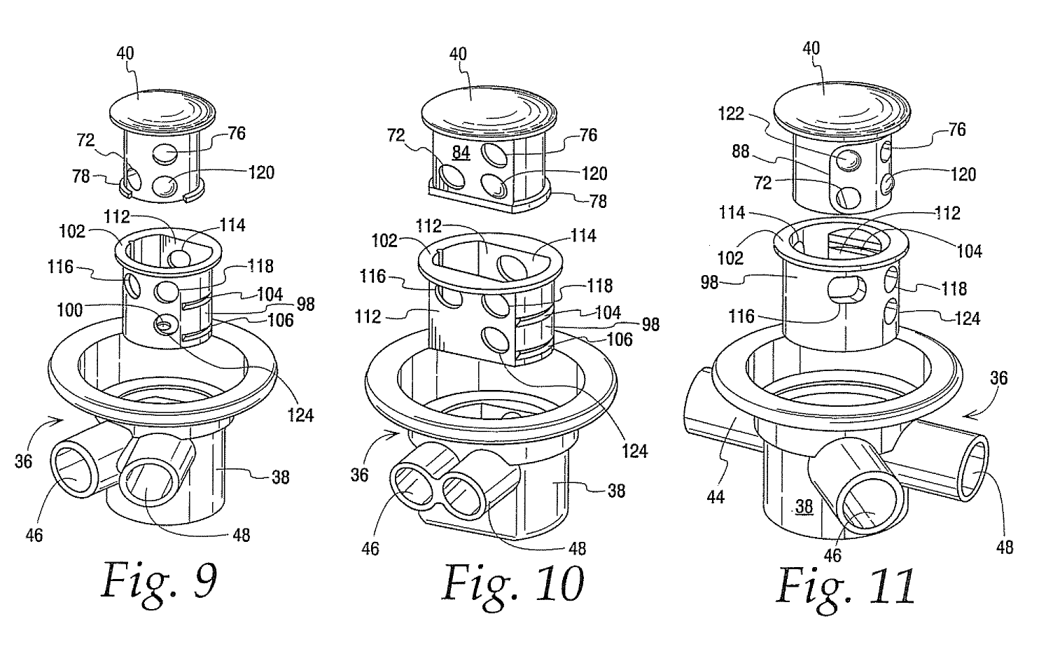

One manner of addressing these concerns is to provide a body formed of PVC and a separate insert formed of a more rigid material that is adapted to withstand deformation during steam sterilization, such as polycarbonate or stainless steel. For example, FIGS. 9-11 show various flow controllers 36 incorporating differently configured inserts 98 interposed between the body 38 and the associated actuator member 40. At its most basic, the insert 98 is a generally cup-shaped element that is immovably received within the body cavity 42 and effectively acts as an inner layer of the body. There is preferably a relatively tight fit between the insert 98 and the cavity 42, 42a, so the insert 98 may be provided with a bottom aperture 100 (FIG. 9) to vent any air trapped between the insert 98 and the cavity 42, 42a during placement. The insert 98 may also include a top flange 102 adapted to bear against the annular seat 60 of the body 38 when the insert 98 is fully inserted.

Once placed into the cavity, the insert 98 may be held in place by any of a number of means, for example by a latching system. The insert 98 may have at least two slots 104 and 106 (FIG. 17), while FIG. 12 illustrates a body cavity 42a having a matching rib or latch 108 and 110 for each slot 104 and 106. The insert 98 is oriented to align a flat wall 112 thereof with the flat wall 82 of the cavity 42a, and then it is inserted until the latches 108 and 110 are received by the slots 104 and 106, respectively. The latches 108 and 110 may provide a ratcheting effect, such that the insert 98 cannot be removed once the latches 108 and 110 are received by the slots 104 and 106.

According to another manner of fixedly securing the insert within the body cavity, the insert is comprised of a material adapted to bond to the body during steam sterilization. Polycarbonate is a preferred insert material, because it is sufficiently rigid to resist deformation, but may also become tack-bonded to a PVC body during steam sterilization. Preferably, the latching mechanism is provided to secure the body and insert during the initial stages of manufacture, with the two becoming bonded together during steam sterilization to assure fixation.

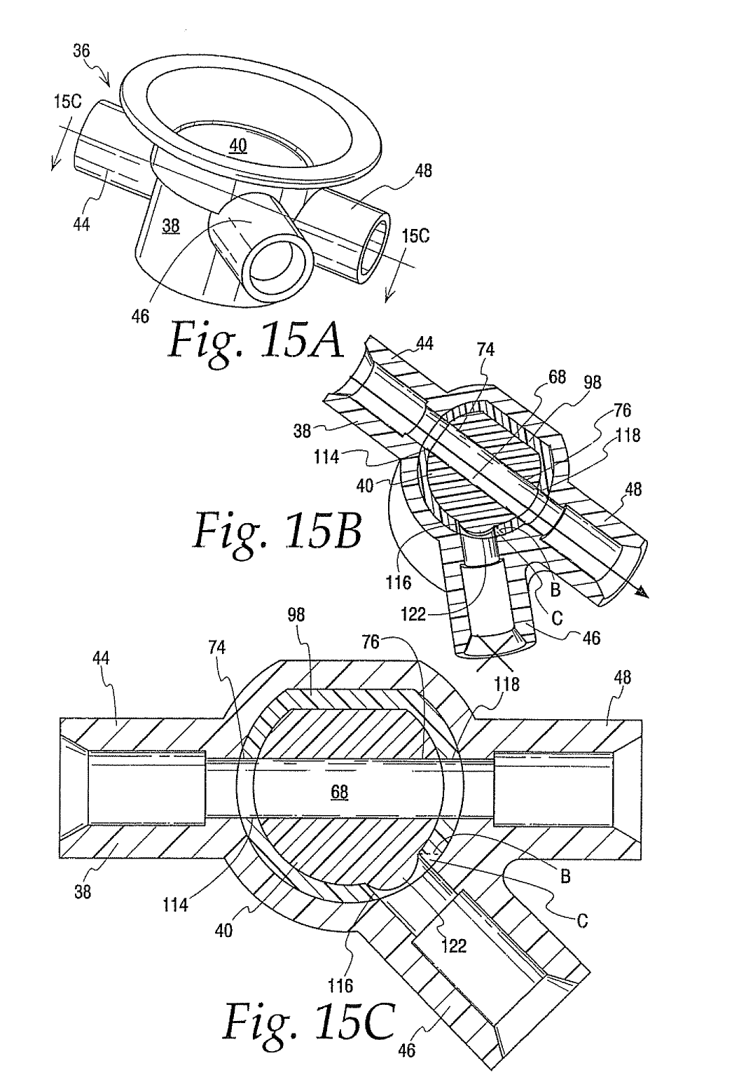

As shown in FIGS. 9-11, the insert 98 includes an inlet hole 114 and two outlet holes 116 and 118 corresponding to the fluid inlet 44 and fluid outlets 46 and 48 of the body 38. Hence, an actuator member 40 received in the insert 98 will operate according to the above description, except that the flow paths 66 and 68 are aligned with the inlet hole 114 and outlet holes 116 and 118 of the insert 98, rather than being directly aligned with the fluid inlet 44 and fluid outlets 46 and 48 of the body 38. For example, FIGS. 14A-15C illustrate the operation of the flow controller 36 of FIG. 11. In the first position (FIGS. 14A-14C), the fluid inlet 44, inlet hole 114, and first fluid entrance 70 are aligned to allow flow into the first flow path 66. At the downstream portion of the first flow path 66, the first fluid outlet 46, first outlet hole 116, and first fluid exit 72 are aligned to allow flow out of the flow controller 36. The actuator member 40 is moved to the second position (FIGS. 15A-15C) to misalign the fluid inlet 44 and the first flow path 66. In the second position, the fluid inlet 44, inlet hole 114, and second fluid entrance 74 are aligned to allow flow into the second flow path 68. At the downstream portion of the second flow path 68, the second fluid outlet 48, second outlet hole 118, and second fluid exit 76 are aligned to allow flow out of the flow controller 38.

The first outlet hole 116 is shown in FIGS. 14B, 14C, 15B, and 15C with an adjacent broken line indicated at “B.” Preferably, the first outlet hole 116 is defined by a bore coaxial with the downstream portion of the first flow path 66 (FIGS. 14B and 14C), but it may simplify molding to provide a bore defined in part by broken line “B,” because such a bore is parallel to the bore defining the second outlet hole 118, thereby minimizing the number of axes during molding. While such a bore simplifies manufacture, it also results in a small triangular cavity “C,” which may create the risk of blood stagnation and coagulation. However, it has been found that the triangular cavity “C” is sufficiently small and, if the first flow path 66 is associated with flow to a sample pouch, the duration of the initial flow is sufficiently minor that the risk of coagulation is acceptably remote.

The latching systems of the embodiments including an insert may operate similarly to the latching system described previously with regard to the embodiment of FIGS. 3-8. The actuator member 40 is provided with a rib or latch 78 (FIG. 16) and the insert 98 is provided with upper and lower slots 104 and 106 (FIG. 17). In the first position, the latch 78 sits in the upper slot 104 of the insert 98 (FIG. 18). When the actuator member 40 is moved to the second position, the latch 78 moves into a lower slot 106 of the insert 98. To prevent the actuator member 40 from moving to the first position from the second position, the latch 78 may interact with the lower slot 106 in a ratcheting manner to prevent retraction. As previously described herein, the body 38 may also include latches 108 and 110 (FIG. 12) adapted to seat within the insert slots 104 and 106, respectively, so the insert 98 is preferably sufficiently thick to allow a slot 104, 106 to simultaneously receive an actuator member latch 78 and a body cavity latches 108, 110.

The actuator member 40 of FIG. 11 is illustrated with a lower sealing bump or projection 120 positioned below the second fluid exit 76 and an upper sealing bump 122 positioned above the first fluid exit 72, each projecting convexly from the curved wall. As shown in FIGS. 14B and 14C, leakage through the second fluid outlet 48 in the first position is further prevented by the lower sealing bump 120 extending into the second outlet hole 118. In the second position (FIGS. 15B and 15C), leakage through the first fluid outlet 46 is further prevented by the upper sealing bump 122 extending into the first outlet hole 116. The insert 98 preferably includes a bump-receiving opening 124 (FIG. 11) below the second outlet hole 118, adapted to receive the lower sealing bump 120 when the actuator member 40 is moved to the second position. It should be understood that the actuator member may be provided with only one, rather than two sealing bumps, and that the sealing bumps and bump-receiving opening may be incorporated into a flow controller according to the embodiment of FIGS. 3-8. Further the sealing bumps may be used instead of latches to unidirectionally secure the actuator member in the first and second positions.

Numerous variations may be incorporated into the described flow controllers without departing from the scope of the present invention. For example, rather than being comprised of a rigid material, the insert may be formed of a more pliant material and used in combination with a more rigid body. Alternatively, the insert may have a layered composition, preferably with a rigid outer layer 126 and a pliant inner layer 128, as shown in FIG. 17. According to one embodiment, a layered insert has a softer inner layer formed of, for example, Cawiton SEBS or polyisoprene or santoprene, and a more rigid outer layer formed of polycarbonate or a metal or ceramic material. In particular, a composite insert comprising a Cawiton SEBS layer and a polycarbonate layer may be preferred, as those materials may be joined by bonding the layers together at a high mold temperature, plus the polycarbonate will become tack bonded to a PVC body during steam sterilization.

A composite implant may be preferred, because the rigid layer prevents deformation during steam sterilization, while the pliant layer forms a tight seal with the actuator member without requiring precise design tolerances. If a pliant insert, or one having a pliant inner layer, is provided, then preferably the actuator member is comprised of a more rigid material having a low coefficient of friction, such as polypropylene. Preferably, the areas surrounding the slots of a composite insert are substantially devoid of the softer material, to provide a more secure latching mechanism and more pronounced tactile and/or audible feedback when the actuator member is moved to the second position.

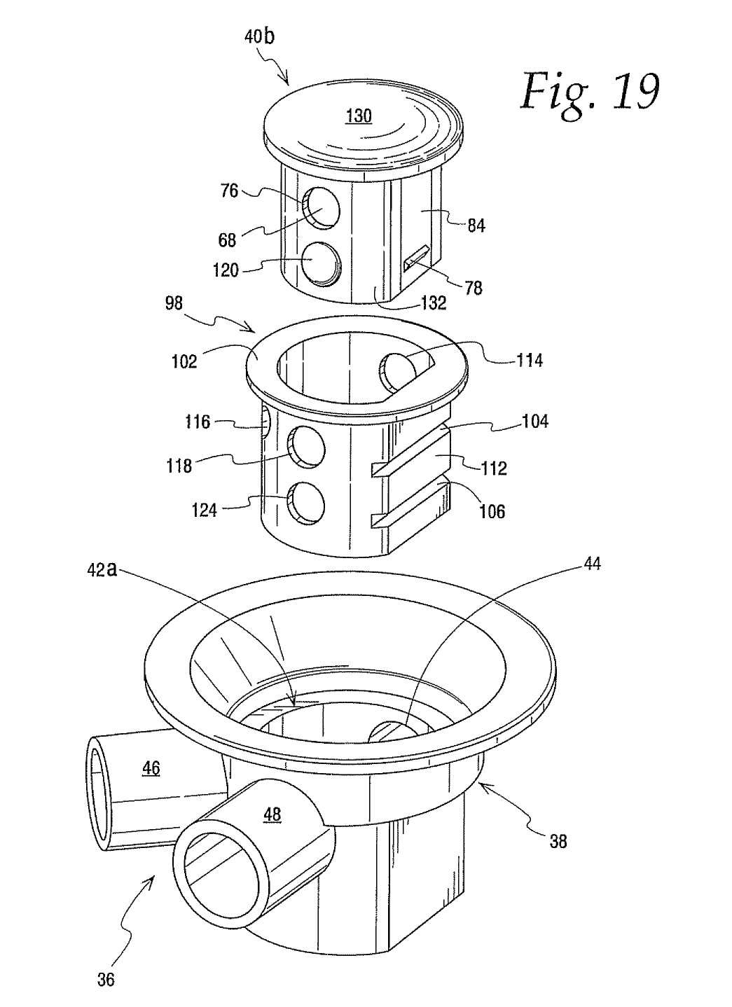

As with the insert, the actuator member may be a composite piece having a rigid layer or portion and a pliant layer or portion. For example, FIG. 19 illustrates an actuator member 40b having a rigid body or core 130 and a curved wall surrounded by a pliant layer 132. The composite actuator member 40b of FIG. 19 is preferably used with a relatively rigid body or, if provided, a relatively rigid insert. In the illustrated embodiment, the flat wall 84 and latch 78 of the actuator member 40b are substantially free of the pliant material, to more securely fit with grooves of the body cavity (not illustrated) or the slots 104 and 106 of the insert 98 and provide enhanced tactile and/or audible feedback when moved to the second position.

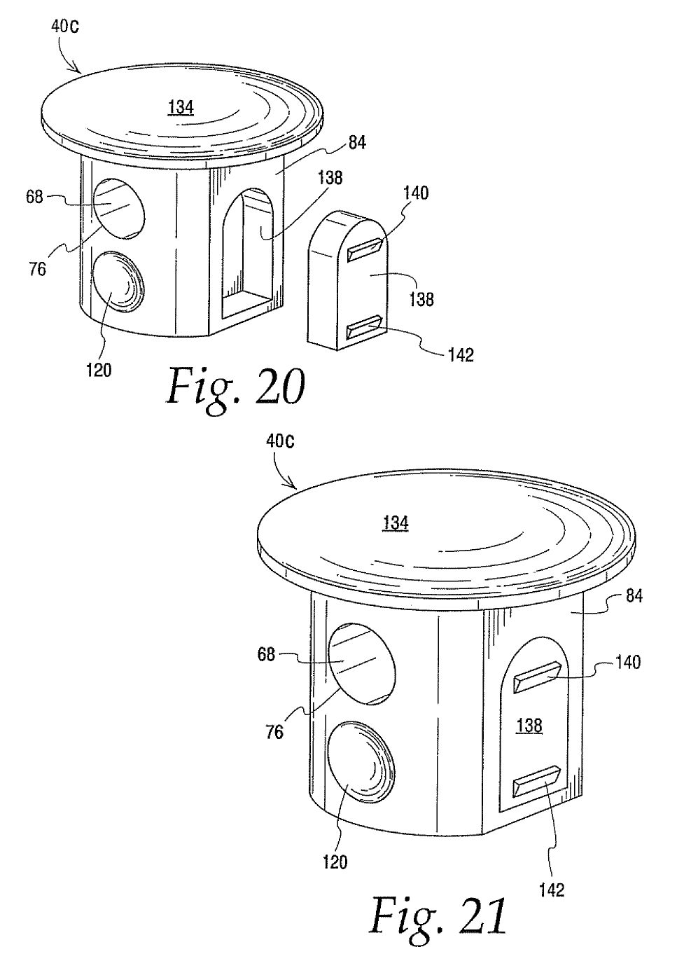

FIGS. 20-23 illustrate another embodiment of a composite actuator member 40c. In this embodiment, the actuator member body 134 is comprised of a relatively pliant material and defines a latch niche 136 (FIGS. 20 and 21) The latch niche 136 is adapted to receive a separate latch member 138 comprised of a relatively rigid plastic, metallic, or ceramic material. While FIGS. 20 and 21 illustrate an actuator member body 134 having a single latch niche 136, it may be preferred to include a second latch niche spaced from the first to receive a second latch member (not illustrated) to allow the actuator member 40c to seat more evenly and discourage “rocking” during movement to the second position.

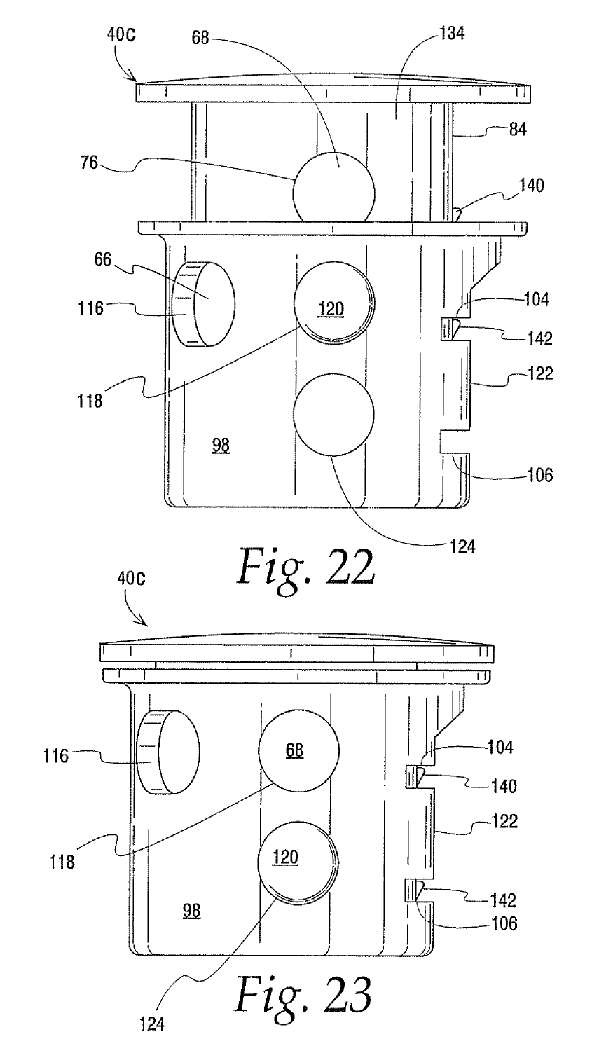

The illustrated latch member 138 includes an upper latch 140 and a lower latch 142 adapted to interact with body grooves or, as shown in FIGS. 22 and 23, insert slots 104 and 106. In the first position (FIG. 22), the lower latch 142 is seated in the upper insert slot 104, with the upper latch 140 some distance above the top of the insert 98. When the actuator member 40c is moved to the second position (FIG. 23), the lower latch 142 moves into the lower insert slot 106 and the upper latch 140 moves into the upper insert slot 104. Such an embodiment may be preferred, because the soft actuator member body 134 compresses to allow the rigid latches 140 and 142 to resiliently yield inwardly during movement to the second position, before springing back to seat within the slots 104 and 106, respectively. It will be appreciated that the provision of a second latch enhances the tactile and/or audible feedback when the actuator member is moved to the second position and further enhances the unidirectional latching safety feature to prevent the actuator member from being retracted from the second position to the first position. Of course, a second latch may be incorporated into an actuator member comprised as a single molded piece and is not limited to composite actuator members.

According to another embodiment, the actuator member has a single flow channel instead of a plurality of distinct channels. For example, FIGS. 24-27 illustrate a flow controller 144 having a body 146 defining a cavity 148, a fluid inlet 150, and two fluid outlets 152 and 154. It will be seen that, in contrast to the embodiments of FIGS. 1-23, it may be preferred for the fluid outlets 152 and 154 of the flow controller 144 to be vertically spaced from each other, rather than angularly separated

In the illustrated embodiment, the first fluid outlet 152 is substantially non-coaxial with the fluid inlet 150, whereas the second fluid outlet 154 is substantially coaxial with the fluid inlet 150. In accordance with the foregoing description of the embodiments of FIGS. 1-23, it has been found that the risk of stagnation and coagulation is minimized by moving blood through a substantially linear flow path. Thus, the non-coaxial first fluid outlet 152 may be associated with an output zone receiving a minor amount of blood, such as a sample pouch 26 (FIG. 2), while the coaxial second fluid outlet 154 may be associated with an output zone receiving a greater amount of blood, such as a main collection container 28 (FIG. 2).

The flow controller 144 includes an actuator member 156 at least partially received within the cavity 148 and movable from a first position (FIG. 25) to a second position (FIG. 26). The actuator member 156 defines a single flow channel 158 having a fluid entrance 160 and a fluid exit 162. In the first position, the fluid entrance 160 is adjacent to the fluid inlet 150 and the fluid exit 162 is adjacent to the first fluid outlet 152, thereby allowing fluid communication between the fluid inlet 150 and the first fluid outlet 152. Fluid flow between the fluid inlet 150 and the second fluid outlet 154 is substantially prevented in the first position.

When a sufficient amount of fluid has been passed through the first fluid outlet 152, the actuator member 156 is advanced farther into the cavity 148 by the user. Typically, this is accomplished by the user gripping the body 146, which may be provided with a finger grip 164, and pressing the actuator member 156 with his/her thumb. The actuator member 156 may be adapted to contact a closed end of the cavity 148 after traveling a certain distance to define a stopping point at the second position. In the second position (FIG. 26), the fluid entrance 160 remains adjacent to the fluid inlet 150, while the fluid exit 162 is moved away from the first fluid outlet 152 to be adjacent to the second fluid outlet 154, thereby allowing fluid communication between the fluid inlet 150 and the second fluid outlet 154. Fluid flow between the fluid inlet 150 and the first fluid outlet 152 is substantially prevented in the second position. The flow controller may include latches 78 (FIG. 27) and a latching system, as described herein with respect to the embodiments of FIGS. 1-23, to prevent movement of the actuator member from the second position to the first position.

Preferably, the actuator member 156 is non-rotatable with respect to the body 146, to prevent misalignment of the flow channel 158. This may be achieved by incorporating a keying feature, such as a projection or flat wall (not illustrated), into a cylindrical actuator member or providing a substantially non-cylindrical actuator member, such as the box-shaped actuator member 156a of FIG. 27. The cavity of the body is preferably shaped to conform to the shape of the actuator member to cooperate therewith in preventing relative rotation.

It will be seen that the fluid entrance 160 is substantially larger than the fluid inlet 150 and that the fluid exit 162 is substantially larger than each of the fluid outlets 152 and 154. In one embodiment, the fluid entrance may be at least approximately 200% larger than the fluid inlet, and the fluid exit may be at least approximately 200% larger than each of the fluid outlets. The exact size and spacing of the inlet 150 and outlets 152 and 154 may vary according to a number of factors, including the nature of the tubing leading to the fluid source and collection containers, so the relative size of the fluid entrance 160 and exit 162 may similarly vary to cooperate with the particular housing design. Preferably, there is a direct correlation between the relative size of the fluid entrance 160 and exit 162 and the spacing between the fluid outlets 152 and 154.

The oversized fluid entrance 160 allows the flow channel 158 to remain open to the fluid inlet 150 in both the first and second positions, while the oversized fluid exit 162 allows the flow channel 158 to switch between communication with the first fluid outlet 152 in the first position (FIG. 25) and the second fluid outlet 154 in the second position (FIG. 26). This switching action may be achieved by a generally Z-shaped flow channel 158, as shown in FIGS. 25 and 26. The vertical extent of the fluid exit 162 and the vertical separation between the fluid outlets 152 and 154 are preferably selected to close flow through the second fluid outlet 154 in the first position (FIG. 25) and through the first fluid outlet 152 in the second position (FIG. 26).

The body 146 may be provided with a sanitary seal or membrane 96 bonded to the finger grip 164 that covers the cavity 148 and encloses the actuator member 156, 156a (FIGS. 25 and 26) to create a sanitary, closed system. Preferably, the membrane 96 is sufficiently deformable to flex and allow the actuator member 156, 156a to be moved from the first position to the second position. PVC is a suitable material for the membrane 96, but other materials may be used without departing from the scope of the present invention.

According to another manner of providing a sanitary, closed system, the flow controller 144 may include at least one gasket or sealing member 166 between the actuator member 156, 156a and the body 146 (FIG. 24), The sealing member 166 is preferably positioned to be at a vertical elevation between the first fluid outlet 152 and the open end of the cavity 148 when the actuator member 156, 156a is received within the cavity 148. If the actuator member is substantially cylindrical (FIGS. 24-26), the sealing member 166 may comprise an o-ring maintained within a circumferential channel (not illustrated), such that the sealing member 166 moves with the actuator member 156 from the first position to the second position. Alternatively, the sealing member may be otherwise fixed to the actuator member to permit it to move therewith. According to yet another embodiment, the sealing member may be fixed to an interior portion of the cavity and be stationary with respect to the movable actuator member.

To improve mobility of the actuator member from the first position to the second position, all or a portion of the exterior surface of the actuator member and/or all or a portion of the interior surface of the body cavity (or insert if provided) may be treated with a lubricant material. The suitability of a particular lubricant material will vary according to the materials comprising the flow controller. For example, if the lubricant material is to be applied to an elastomeric silicone component, a polymer cross-linking coating, such as LSR Top Coat from GE Advanced Materials-Silicones of Waterford, N.Y., may be used. Other lubricating and friction-reducing means may also be incorporated without departing from the scope of the present invention.

From time to time, the terms “inlet,” “outlet,” “entrance,” and “exit” were used herein to refer to components of flow controllers according to the present invention. These terms refer to the orientation of the components in applications involving a single fluid being delivered to two separate locations, such as blood from a donor being delivered to a sample pouch and a main collection container. However, flow controllers according to the present invention may be used in applications where fluid pass into the flow controller through one of the “outlets” and leaves the flow controller through the “inlet.” For example, a first fluid may flow through the first fluid outlet 46, 152 and out the fluid inlet 44, 150, and then the actuator member 40, 156 may be moved to the second position to allow a second fluid to flow through the second fluid outlet 48, 154 and out the fluid inlet 44, 150. The reconstitution or sequential mixing of certain fluid medicaments are exemplary of applications requiring such flow. Hence, the terms “inlet,” “outlet,” “entrance,” and “exit” are not to be understood as limiting the described flow controllers to particular applications or as limiting the scope of the claims.

It will be understood that the embodiments described above are illustrative of some of the applications of the principles of the present invention. Numerous modifications may be made by those skilled in the art without departing from the spirit and scope of the invention, including those combinations of features that are individually disclosed or claimed herein. For these reasons, the scope of the invention is not limited to the above description but is as set forth in the following claims.

1. A flow controller comprising:
a body defining a cavity, said body comprising a fluid inlet, a first fluid outlet, and a second fluid outlet; and
an actuator member at least partially received within the cavity and defining a first flow channel and a second flow channel, wherein
the actuator member is adapted for at least substantially non-rotational movement from a first position to a second position within said cavity,
the first flow channel allows for fluid communication between the fluid inlet and the first fluid outlet in said first position,
the second flow channel allows for fluid communication between the fluid inlet and the second fluid outlet in said second position, and
the actuator member is prevented from moving from said second position to said first position.

2. The flow controller of claim 1, further comprising means for preventing movement of the actuator member from the second position to the first position.

3. The flow controller of claim 1, wherein said flow channels are substantially comprised of a non-compressible material.

4. The flow controller of claim 1, whereby in said first position fluid communication between the fluid inlet and second fluid outlet is substantially prevented, and in said second position fluid communication between the fluid inlet and the first fluid outlet is substantially prevented.

5. The flow controller of claim 1, wherein said body is substantially comprised of a first material and said actuator member is substantially comprised of a second material, and wherein one of said first and second materials is more rigid than the other of said first and second materials.

6. The flow controller of claim 1, further comprising a sanitary seal substantially enclosing the actuator member within the cavity.

7. The flow controller of claim 1, wherein the fluid inlet and the fluid outlets define a flow plane, and wherein the actuator member is linearly movable from the first position to the second position along a path generally perpendicular to the flow plane.

8. The flow controller of claim 1, wherein said actuator member is comprised of a first material and a separate second material, wherein one of said first and second materials is more rigid than the other of said first and second materials.

9. The flow controller of claim 1, wherein said fluid inlet and said second fluid outlet are substantially coaxial, and wherein said first fluid outlet is substantially non-coaxial with said fluid inlet.

10. The flow controller of claim 1, wherein said fluid inlet and said fluid outlets are substantially parallel and non-coaxial with each other.

11. The flow controller of claim 1, further comprising a convex bump of the actuator member, wherein said bump is adapted to extend into the second fluid outlet when the actuator member is in the first position.

12. The flow controller of claim 10, further comprising a second convex bump of the actuator member, wherein said second bump is adapted to extend into the first fluid outlet when the actuator member is in the second position

13. The flow controller of claim 1, further comprising a flat wall of the cavity and a flat wall of the actuator member, wherein the flat walls are aligned for the cavity to receive the actuator member.

14. The flow controller of claim 1, further comprising a tactile and/or audible indication when the actuator is moved to the second position.

15. A flow controller comprising:
a body defining a cavity, said body comprising a fluid inlet, a first fluid outlet, and a second fluid outlet;
a generally cup-shaped insert received within the cavity, said insert comprising an inlet hole aligned with the fluid inlet, a first outlet hole aligned with the first fluid outlet, and a second outlet hole aligned with the second fluid outlet; and
an actuator member at least partially received within the insert for movement from a first position to a second position within said insert, whereby in said first position the actuator member allows for fluid communication between the fluid inlet and the first fluid outlet, and in said second position the actuator member allows for fluid communication between the fluid inlet and the second fluid outlet.

16. The flow controller of claim 15, further comprising means for preventing movement of the actuator member from the second position to the first position.

17. The flow controller of claim 15, wherein said insert is substantially comprised of a material adapted to bond to the body upon steam sterilization.

18. The flow controller of claim 15, wherein said insert is substantially comprised of a material adapted to resist deformation upon steam sterilization.

19. The flow controller of claim 15, wherein said body is substantially comprised of a first material and said insert is substantially comprised of a second material, and wherein one of said first and second materials is more rigid than the other of said first and second materials.

20. The flow controller of claim 18, wherein said insert is substantially comprised of stainless steel.

21. The flow controller of claim 15, wherein said insert is comprised of an inner layer and an outer layer, and wherein said outer layer is more rigid than said inner layer

22. The flow controller of claim 15, whereby in said first position fluid communication between the fluid inlet and second fluid outlet is substantially prevented, and in said second position fluid communication between the fluid inlet and the first fluid outlet is substantially prevented.

23. The flow controller of claim 15, wherein said fluid inlet and said second fluid outlet are substantially coaxial, and wherein said first fluid outlet is substantially non-coaxial with said fluid inlet.

24. The flow controller of claim 15, further comprising a convex bump of the actuator member and a bump-receiving opening below the second outlet hole of the insert, wherein said bump is adapted to extend into the second outlet hole when the actuator member is in the first position and to extend into the bump-receiving opening when the actuator member is in the second position.

25. The flow controller of claim 24, further comprising a second convex bump of the actuator member, wherein said second bump is adapted to extend into the first fluid outlet when the actuator member is in the second position.

26. The flow controller of claim 15, wherein the actuator member is adapted for at least substantially non-rotational movement from the first position to the second position.

27. The flow controller of claim 26, further comprising a flat wall of the insert and a flat wall of the actuator member, wherein the flat walls are aligned for the insert to receive the actuator member.

28. A fluid processing set comprising:
a first collection container;
a second collection container; and
a flow controller comprising
a body defining a cavity, said body comprising a fluid inlet, a first fluid outlet communicating with the first collection container, and a second fluid outlet communicating with the second collection container, and
an actuator member at least partially received within the cavity and defining a first flow channel and a second flow channel, wherein
the actuator member is adapted for at least substantially non-rotational movement from a first position to a second position,
the first flow channel allows for fluid communication between the fluid inlet and the first fluid outlet in said first position,
the second flow channel allows for fluid communication between the fluid inlet and the second fluid outlet in said second position, and
the actuator member is prevented from moving from said second position to said first position.

29. The fluid processing set of claim 28, wherein said fluid inlet and said second fluid outlet are substantially coaxial, and wherein said first fluid outlet is substantially non-coaxial with said fluid inlet.

30. The fluid processing set of claim 28, wherein said second collection container is adapted to receive a greater amount of fluid than said first collection container.

31. The fluid processing set of claim 30, wherein said first collection container comprises a blood sample pouch, and wherein said second collection container comprises a main collection container.

32. The fluid processing set of claim 31, wherein said first collection container defines an internal chamber and further includes an internal flow path that extends into said chamber.

33. The fluid processing set of claim 28, further comprising a Y-type access site in fluid communication with said flow controller and said first collection container.

34. The fluid processing set of claim 33, wherein said Y-type access site is adapted to receive a tube holder.

35. The fluid processing set of claim 28, further comprising means for preventing movement of the actuator member from the second position to the first position.

36. The fluid processing set of claim 28, whereby in said first position fluid communication between the fluid inlet and second fluid outlet is substantially prevented, and in said second position fluid communication between the fluid inlet and the first fluid outlet is substantially prevented.

37. The fluid processing set of claim 28, wherein the fluid inlet and the fluid outlets define a flow plane, and wherein the actuator member is linearly movable from the first position to the second position along a path generally perpendicular to the flow plane.

38. The fluid processing set of claim 28, further comprising a generally cup-shaped insert received within the cavity, said insert comprising an inlet hole aligned with the fluid inlet, a first outlet hole aligned with the first fluid outlet, and a second outlet hole aligned with the second fluid outlet, wherein the actuator member is at least partially received within the insert for movement from a first position to a second position within said insert, whereby in said first position the first flow channel allows for fluid communication between the fluid inlet and the first fluid outlet, and in said second position the second flow channel allows for fluid communication between the fluid inlet and the second fluid outlet.

39. A method of collecting at least two quantities of a fluid from a fluid source, comprising:
providing a first collection container and a second collection container;
providing a flow controller body having a fluid inlet, a first fluid outlet communicating with the first collection container, and a second fluid outlet communicating with the second collection container;
providing an actuator member defining a first flow channel and a second channel separate from said first channel, said actuator member movably received by the body;
introducing flow of said fluid to the fluid inlet of the flow controller body with the actuator member in a first position within the flow controller body, thereby directing the flow through said first flow channel and said first fluid outlet to said first collection container;
moving the actuator member from the first position to a second position within the flow controller body without substantial rotational movement, thereby directing the blood flow through said second flow channel and said second fluid outlet to said second collection container; and
preventing movement of the actuator member from the second position to the first position.

40. The method of claim 39, wherein said fluid is blood.

41. The method of claim 39, wherein said introducing flow includes preventing flow to the second collection container.

42. The method of claim 39, wherein said moving the actuator member includes preventing flow to the first collection container.

43. The method of claim 39, wherein said introducing flow includes directing an amount of flow to the first collection container and wherein said moving the actuator member includes directing a greater amount of flow to the second collection container.

44. The method of claim 39, wherein said providing an actuator member includes providing a sanitary seal substantially enclosing the actuator member within the body.
